(19) United States
(12) Patent Application Publication
(10) Pub. No.: US 2010/0274355 A1
(43) Pub. Date: Oct. 28, 2010

(54) Bone-tendon-bone assembly with cancellous allograft bone block having cortical end portion

(76) Inventors: David A. McGuire, Anchorage, AK (US); Anton J. Steiner, Wharton, NJ (US); Stephen D. Hendricks, Anchorage, AK (US); Stuart Archer, Wall Township, NJ (US); Knight David I., Perth Amboy, NJ (US)

Correspondence Address: JOHN S. HALE;GIPPLE & HALE, 6665-A OLD DOMINION DRIVE, MCLEAN, VA, 22101, US

(21) Appl. No.: 12/656,125

(22) Filed: Jan. 19, 2010

Related U.S. Application Data
(60) Provisional application No. 61/202,029, filed on Jan. 21, 2009.

(51) Int. Cl. A61F 2/08

(52) U.S. Cl. 623/13.14

(57) ABSTRACT

The invention is directed toward a sterile bone-tendon-bone assembly with two allograft bone blocks constructed with a cancellous portion and a cortical end portion. Each bone block has an outer curved surface with two opposing longitudinal arcuate grooves cut into the exterior surface which will allow a tendon replacement member to be wrapped around the bone block. The second bone block being in reversed orientation to the first bone block.

### RELATED APPLICATION

The present application is related to and claims priority from U.S. Provisional Patent Application No. 61/202,029 filed Jan. 21, 2009.

### FIELD OF INVENTION

The present invention is generally directed toward a surgical implant assembly and more specifically is a shaped block implant assembly with each block of the assembly being constructed with a cortical end portion and a cancellous body portion defining arcuate channels which receive a replacement member for ligament repair.

### BACKGROUND OF THE INVENTION

Failed ligaments, such as the anterior or posterior cruciate ligaments in the knee joint, significantly limit physical activity and potentially cause chronic knee problems. The anterior cruciate ligament (hereinafter ACL) and the posterior cruciate ligament (PCL) to a lesser extent are often torn during sports related injuries or as result of traumatic stresses. Ligament reconstruction with allograft and autograft tissue has been shown to improve joint function and provide long term improvement in restoration of physical activity. A common surgical method of repair of an ACL is harvesting a patient's patellar tendon with bone blocks from the tibia and patella. The bone-patellar tendon-bone implant offers several advantages, including high initial tensile strength, stiffness, proper length, rigid fixation and direct bone-to-bone incorporation.

The ACL of the knee functions to resist anterior displacement of the tibia from the femur at all flexion positions. The ACL also resists hyper-extension and contributes to rotational stability of the fully extended knee during internal and external tibial rotation. Structurally, the ACL attaches to a depression in the front of the intercondylar eminence of the tibia extending posteosuperior to the medial wall of the lateral femoral condyle. Partial or complete tears of the ACL are very common, comprising about 100,000 outpatient procedures in the U.S. each year. The preferred treatment of the torn ACL is ligament reconstruction, using a bone-ligament-bone autograft. Cruciate ligament reconstruction has the advantage of immediate stability and a potential for immediate vigorous rehabilitation. The disadvantages to autogenous ACL reconstruction are significant: for example, normal anatomy is disrupted when the patellar tendon or hamstring tendons of the patient are used for the reconstruction; placement of intraarticular hardware is required for ligament fixation; and anterior knee pain frequently occurs. Moreover, recent reviews of cruciate ligament reconstruction indicate an increased risk of degenerative arthritis with intraarticular ACL reconstruction in large groups of patients.

A second method of treating ACL injuries, referred to as “primary repair”, involves suturing the torn structure back into place. Primary repair has the potential advantages of a limited arthroscopic approach, minimal disruption of normal anatomy, and an out-patient procedure under a local anesthetic. The potential disadvantage of primary cruciate ligament repair is that over the long term, ACL repairs do not provide stability in a sufficient number of patients, and that subsequent reconstruction may be required at a later date. The success rate of such primary anterior cruciate ligament repair can range from 25% to 75%.

The autogenous patellar tendon is an excellent replacement member providing proper tendon length and bone blocks that are fully osteointegrated without immunological rejection. Unfortunately harvesting autogenous bone-tendon-bone (hereinafter B-T-B) also has a number of adverse risks and effects, including donor morbidity (pain), patellar fracture, tendon rupture and degeneration of the patellofemoral articular surface. As an alternate to autogenous graft tissue, synthetic materials have previously received FDA approval. In this regard polyester braids, steel wire and PTFE (GORE-TEX) have been used surgically. All of these materials have failed to integrate into the bone and have finite bending cycles resulting in the tendon's inability to sustain the tensile and torsional loads applied to the knee in normal usage. Nearly all of these synthetic repairs have been revised with autogenous and/or allograft tissue.

There is a limited supply of allograft bone-patellar tendon-bone (B-PT-B) tissue due in large part to the number of donors that qualify according to the selective donor acceptance criteria. As a result of the limited number of available grafts there is a demand for such grafts which exceeds supply.

The use of substitute bone tissue dates back well over 100 years. Since that time, research efforts have been undertaken toward the use of materials which are close to bone in composition to facilitate integration of bone grafts. Development has taken place in the use of grafts of a mineral nature such as corals, hydroxyapatites, ceramics or synthetic materials such as biodegradable polymer materials. Surgical implants should be designed to be biocompatible in order to successfully perform their intended function. Biocompatibility may be defined as the characteristic of an implant acting in such a way as to allow its therapeutic function to be manifested without secondary adverse affects such as toxicity, foreign body reaction or cellular disruption.

Human allograft tissue is widely used in orthopaedic, neuro-, maxillofacial, podiatric and dental surgery. The allograft tissue is valuable because it is strong, biointegrates in time with the recipient patient's tissue and can be shaped either by the surgeon to fit the specific surgical defect or shaped commercially in a manufacturing environment. Contrasted to most synthetic absorbable or nonabsorbable polymers or metals, allograft tissue is biocompatible and integrates with the surrounding tissues. Allograft bone occurs in two basic forms; cancellous and cortical. Cancellous bone is a less dense structure than that of cortical bone and, like cortical bone, is comprised of triple helix strands of collagen fiber, reinforced with hydroxyapatite. The cancellous bone includes void areas with the collagen fiber component contributing in part to torsional and tensile strength. Cortical bone is more dense and has higher mineralization without the void areas.

Many devices of varying shapes and forms are fabricated from allograft cortical tissue by machining. Surgical implants such as pins, rods, screws, anchors, plates, intervertebral spacers and bone-tendon-bone blocks have been made and used successfully in human surgery. These pre-engineered shapes are used by the surgeon in surgery to restore defects in bone to the bone's original anatomical shape. At the present time cancellous bone is not generally used for shaped devices such as bone-tendon-bone blocks which are subject to pull out forces.

Allograft bone is a logical substitute for autologous bone. It is readily available and precludes the surgical complications and patient morbidity associated with obtaining autologous bone as noted above. Allograft bone is essentially a collagen fiber reinforced hydroxyapatite matrix containing active bone morphogenic proteins (BMP) and can be provided in a sterile form. The demineralized form of allograft bone is naturally both osteoinductive and osteoconductive and surface demineralization treatment of shaped bone grafts which are load bearing also increases the osteoinductivity of the graft. The demineralized allograft bone tissue is fully incorporated in the patient's tissue by a well established biological mechanism. It has been used for many years in bone surgery to fill the osseous defects previously discussed.

U.S. Pat. No. 5,562,669 issued Oct. 8, 1996 discloses a B-T-B tendon anchor device using autologous bone plugs taken from the cores drilled out from the bone tunnels of the patient or alternatively donor bone, namely allograft bone to make the bone plugs. The linear cylindrical plug member is provided with two longitudinal substantially parallel grooves cut on opposite sides of each bone plug which provide a recess in which the ligament replacement member can be seated. Suture holes located in the grooves are cut through the bone plug for attaching the tendon to the plug as is shown in FIGS. 4a and 4b. The grooves position the tendon equally on both sides of the bone block.

Likewise, U.S. Pat. No. 5,632,748 issued May 27, 1997 discloses a B-T-B tendon anchor device formed of plastic, bone, stainless steel or any other suitable material. The body is tapered and formed with a groove to receive a fixation screw and two curved toothed grooves to hold a tendon which is looped over the device. The fixation groove is provided with threads (FIG. 3) and the tendon grooves are provided with teeth. (FIG. 4). A two piece version having a tongue and groove and stepped mating faces for joinder with two tendon grooves is shown in FIG. 7.

U.S. Pat. No. 6,730,124 issued May 4, 2004 discloses a bone-tendon-bone assembly with a cancellous allograft bone block having a single external groove, an opposing planar surface and a through going central bore. The bone block body is also provided with a plurality of suture holes radially cut through the bone block.

U.S. Pat. No. 7,141,066 issued Nov. 28, 2006 discloses a bone-tendon-bone assembly with a cortical allograft bone block having a external groove, a curved opposite surface and a through going central bore. The external groove has a plurality of suture holes cut through the groove into the central bore of the bone block.

Presently, the bone block systems in B-T-B grafts have been made from cortical bone, cancellous bone or synthetic materials. While cancellous bone has many advantages when used, the cancellous plug cleaves perpendicular to the applied force while the attached surface generally retains contact with the tunnel wall. It (the cancellous bone) is the weak link in grafts. Conversely, an all cortical plug requires tapping, otherwise the threads of the interference screw make surface contact but do not integrate with the plug. The subsequent mode of failure with such cortical plugs is between the screw and the cortical surface of the plug. The cortical bone is the weak link. Tapping the plug intra-operatively, or pretapping when possible resolves this problem but it is very time and technically consuming. Pre-tapping the plug poses an additional problem in that screw thread depth and pitch varies amongst all of the interference screws available to surgeons producing a high risk of mismatch between a pre-tapped plug and available screws.

Previously, the iliac crest has not been used for shaped implant grafts because of the cortical/cancellous composition of the ilium tissue.

### SUMMARY OF THE INVENTION

The present invention is directed to a bone-tendon-bone composite graft of novel construction for use in tendon ligament reconstruction using a cancellous bone block with a cortical end cap defining opposing arcuate channels running the length of the bone block. The allograft bone blocks are pre-machined from ilium tissue taken from the iliac crest to form a solid block with a main cancellous body and a cortical end portion. At least one tendon replacement member, such as a semitendinous, tibialis, gracilis tendon, any other suitably long and strong enough tendon, or a combination of tendons is extended around the bone blocks on the arcuate channels over an end of the bone block and back along the opposing arcuate channel formed on the opposite outer surface of each bone block. The bone blocks are secured in the respective tunnel of the patients bone by an interference screw which is inserted against the outer curved surface of the bone block between the tendon grooves. The tendon replacement member is in turn secured to the two bone blocks by sutures and pre-tensioned on a graft table. The use of the bone-tendon-bone composite graft of the invention results in a reconstructed ligament.

It is object of the invention to utilize a shaped bone implant structure which approximates the mechanical strength characteristics of a natural cortical bone-tendon-bone graft to provide overall strength and initial durability to the structure.

It is another object of the invention to provide a pre-machined bone block having a cancellous portion and a cortical end portion to prevent the ligament member from cleaving the plug axially.

It is also an object of the invention to provide a pre-machined bone derived structure which can effectively promote new bone growth and accelerate healing when implanted into a human.

It is yet another object of the invention to create a bone-tendon-bone assembly which mimics the configuration of natural bone-tendon-bone constructs.

It is an additional object of the invention to use the iliac crest which has previously been discarded during the bone recovery process for shaped allograft implants

It is still another object of the invention to create a bone-tendon-bone assembly which can be easily handled by the physician during surgery which eliminates or significantly reduces the physician from carving the respective bone blocks.

These and other objects, advantages, and novel features of the present invention will become apparent when considered with the teachings contained in the detailed disclosure which along with the accompanying drawings constitute a part of this specification and illustrate embodiments of the invention which together with the description serve to explain the principles of the invention.

### BRIEF DESCRIPTION OF THE DRAWINGS

FIG. 1 is a perspective view of the inventive bone-tendon-bone block implant

FIG. 2 is a side elevational view of the inventive bone-tendon-bone block of FIG. 1;

FIG. 3 is a front elevational view of the inventive bone-tendon-bone block of FIG. 1;

FIG. 4 is a top plan of the bone block of FIG. 1;

FIG. 5 is a top plan view of the bone-tendon-bone assembly using the bone block of FIG. 1 with the tendon replacement member sutured across the tendon member;

FIG. 6 is a side view of the bone-tendon-bone assembly of FIG. 5 showing the tendon ends tied together;

FIG. 7 is a top plan view of the bone-tendon-bone assembly using the bone block of FIG. 1 with the tendon replacement member sutured parallel to the axial alignment of the tendon member; and

FIG. 8 is a side view of the bone-tendon-bone assembly of FIG. 7;

### DETAILED DESCRIPTION OF THE INVENTION

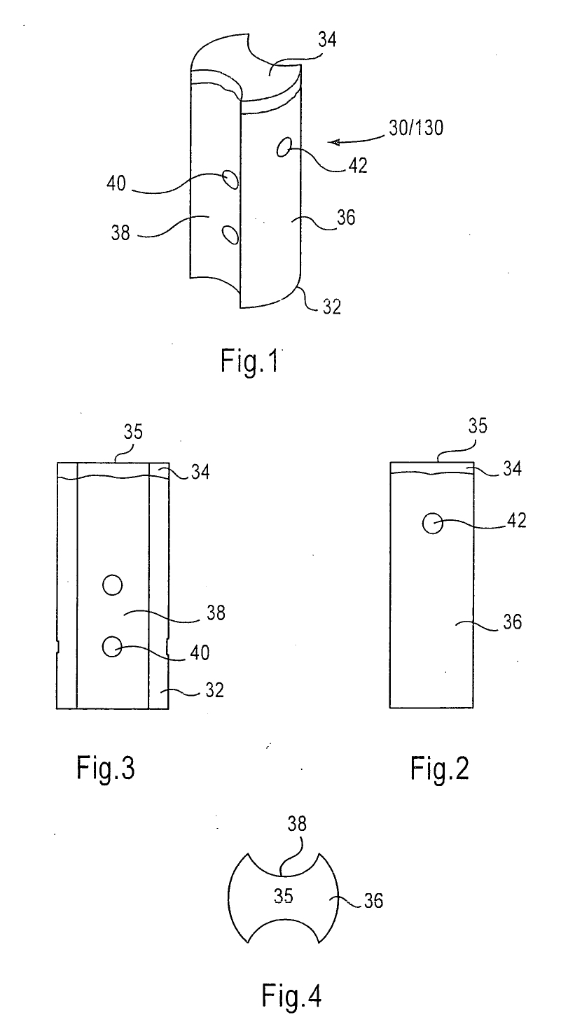

The preferred embodiment and best mode of the present invention is shown in FIGS. 1-8.

As shown in the drawings, a reconstructed bone-tendon-bone (B-T-B) assembly for a knee joint is shown as is well known in the art and which is incorporated by reference in U.S. Pat. Nos. 6,730,124 issued May 4, 2004 and 7,141,066 issued Nov. 28, 2006. The cruciate ligament reconstruction surgical operation can be conducted as an open orthopedic surgery or through arthroscopic surgery. While the description of the invention is primarily directed to knee reconstruction, the present invention can easily be adapted to other joints requiring ligament or tendon replacement.

A number of surgical methods and variation of the same can be used in the tendon reconstructive surgery. Representative methods which are exemplary but not exclusive or limited are referred to as the Lipscom et al. Technique, the Puddu Technique, the Zaricznyj Technique, the Zarins and Rower Techniques and are set forth and fully explained in Chapter 29, Knee Injuries, Campell's Orthopaedics (1998, 9^{th} Ed.) and are incorporated herein by reference. In most B-T-B procedures, anteromedial and distal lateral bores are drilled to give access to the knee joint for these procedures.

In the standard ACL (anterior cruciate ligament) reconstruction, the intercondylar notch is prepared by a rotary shaver inserted through the anteromedial portal performing a notchplasty along the medial aspect of the lateral femoral condyle which may include an accompanying 1 mm to 2 mm roofplasty. The tibial tunnel is prepared by drilling using a externally guided reamer of 8 mm to 12 mm diameter. Guides are used to place the tunnels anatomically, either over a guide wire, or externally guided trephines in the tibia and femoral anatomy. The tibial tunnel entrance is midway between the tibial tubercle and the posterior medial edge of the proximal tibia, approximately three finger widths below the joint line. The exit for the tibia tunnel is the posterior medial footprint of the native ACL. With the knee positioned at 60 degrees of flexion, the guide pin is placed with a suitable guide from the tibial tunnel entrance so that it exits to the center of the selected tibial plateau tunnel aperture posterior medial footprint of the native ACL. Once placed, a cannulated fully fluted reamer is used to drill the tibial tunnel, An alternate method uses externally guided trephines with the tang of the guide placed in the posterior aspect of the tibial plateau just anterior to the PCL such that the resultant posterior wall of the tibial tunnel will exist 6 mm anterior to the over-the-back ridge the PCL rests against. The femoral tunnel is then placed using an endoscopic femoral aimer (EFA) so that its tang is high in the posterior aspect of the intercondylar notch around the 11 o'clock position for the right knee and around the 1 o'clock position for the left knee. The EFA is then used to place the guide wire, depending on the selected tunnel size, from 5 to 7 mm anterior from the over-the-top position placement of the EFA tang. Once the guide wire is inserted and the EFA is removed, a cannulated reamer is drilled over the guide wire to accommodate a bone block of a B-T-B assembly.

The two major bones that meet at the knee joint are the tibia and the femur and bone tunnels are drilled through each of these two major bones by a fully fluted or acorn reamer or coring reamer with a desired diameter. The coring reamer drills out a core of bone from the tibia and the acorn or fully fluted reamer is used to drill the femoral tunnel in a transtibial method by passing it through the reamer produced tibial tunnel forming aligned bone tunnels. The knee is flexed or extended a variable amount in order to properly position the femoral tunnel. The bone debris is evacuated from the knee compartment. Standard deburing and debridement procedures are followed. The graft is passed into position in the femoral tunnel and fixed with an interference screw.

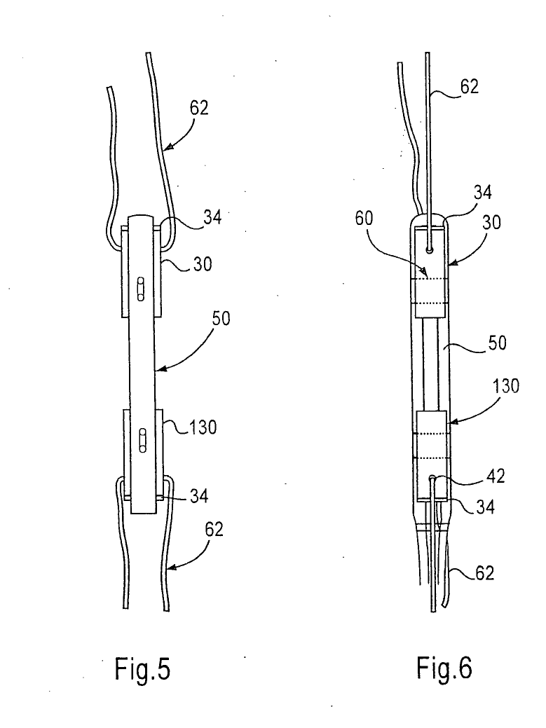

For the purposes of the present invention, relative size relationships will be set forth which should not be constructed as forming specific size limitations. After the bone cores or debris have been drilled out forming bone tunnels which generally range from 9 to 12 mm diameter, an allograft B-T-B assembly with pre-machined bone blocks 30, 130 and an attached treated tendon member(s) 50 is inserted into the bone tunnels by pulling the respective bone blocks into the tunnels via passing sutures 62 with the bone blocks being fixed in the respective femur/tibia tunnel by an interference screw (not shown). The interference screw engages the bone block 30/130 on the outward curved outer surface 36 away from tendon member(s) 50 to hold the tendon member(s) 50 in place. The general OD of the bone block and tendon is around 9-12 mm or the diameter of the bone tunnel allowing the same to be frictionally held in place prior to interference engagement with an interference screw. The interference screw threads engage and compress the cancellous bone increasing the density of the bone and its surface contact with the screw until a pull out strength of over 200 Newtons is reached. As can be seen in the FIGS. 5-8, the sliding bone blocks 30/130 are mounted on tendon members 50 or bundles in a reversed position. This opposite positioning of the bone blocks balances the tendon bundle tension.

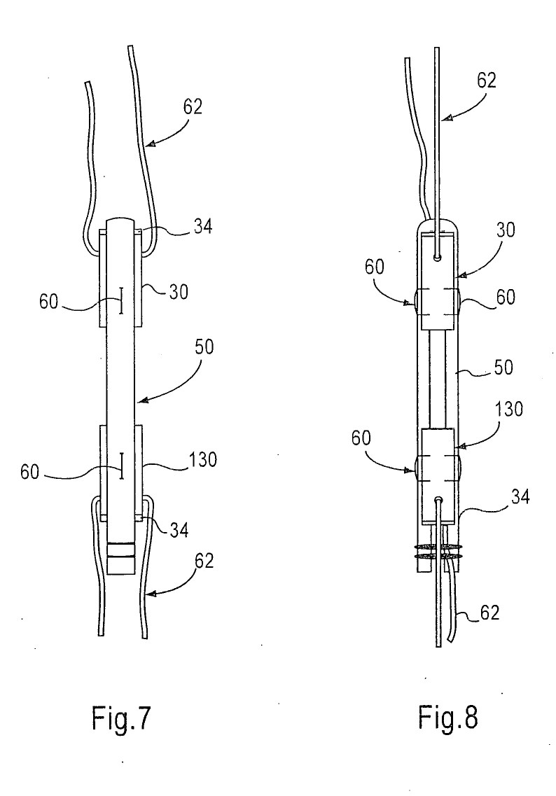

As can be seen in FIGS. 1-4, a bone block body 30 of substantially cylindrical shape is preferably formed of allograft bone with a cancellous bone portion 32 and a cortical bone end portion 34. As noted, the bone block body 30 can be formed from the iliac crest. If desired, the block body can be surface demineralized to a preferred depth of 30 to 80 microns. Alternatively, the body can be formed of xenograft material or synthetic material that is biocompatible and suitably implantable in humans. The curved outer surface 36 of the bone block body has two opposed arcuate channels or grooves 38 which run longitudinally the length of the block to provide a seating surface for seating the looped tendon 50. The distance between the channel bottoms of the opposing grooves preferably ranges from 3.0 mm to 6.0 mm and most preferably ranges from about 3.8 mm to about 4.2 mm. The curved outer surface 36 provides an interference fit with the fixation screw to hold the bone block 30/130 in place in the respective bone tunnel. A plurality of through going suture bores 40 preferably spaced about 6.0 mm apart run through the block body transverse to the longitudinal axis of the block with the end opening located in grooves 38 and at least one passing suture bore 42 runs across the diameter of the block with the ends of the bore being positioned on the curved outer surface 36 of the block with the axis of the bore 42 being transverse to the axis of the suture bores 40. The passing suture bore 42 is positioned near the cortical end portion 34 about 6.0 mm from the planar end surface 35. As previously noted the bone block diameter runs generally from 9 to 12 mm with a corresponding length of 23 to 30 mm, preferably about 23.8 to 24.2 mm depending upon surgeon preference and the diameter of the bone tunnels being used. Suture bores 40 are radially cut through the bone block for attaching the tendon(s) 50 to the bone block 30, 130 via sutures 60 as seen in FIGS. 5-8. In the preferred embodiment, at least two such suture bores 40 are drilled through the bone block with the open ends of the bores 40 being positioned in the bottom of grooves 38. Sutures 60 can be placed in bores 40 and wrapped around the outside of the tendon member 50 as shown in FIGS. 5 and 6 or placed in bores 40 and inserted through the tendon 50 to form a axial positioned loop running along the axis of the tendon as shown in FIGS. 7 and 8. Passing sutures 62 are run through suture bore 42. One suture in the femoral tunnel bone plug is used to pull the assembled composite graft into the desired location in the bone tunnels and the other wire suture inserted into the tibial tunnel plug is used to adjust the assembly tension during tibial plug fixation with an interference screw subsequent to securing the femoral tunnel plug with its interference screw. The sutures may also be used after graft assembly to pretension the graft on a standard graft table.

The assembly is preassembled on a graft table. The lengths of the bone plugs are determined and the inter-compartmental distance from the apertures of the tibial and femoral tunnels are measured and used to calculate the loop length of the ligament replacement member. This distance represents the length of the native ACL. This distance may be measured preoperatively on a lateral view radiograph and accordingly, the graft may be preassembled preoperatively.

The free ends of the ligament member are sutured together to form a loop of the desired length. The desired length of the ligament member when combined with the bone plugs produces a graft, which when inserted into its position within both tunnels, each plug is minimally recessed within each tunnel aperture and thus precludes the bone plug from protruding beyond the aperture into the knee compartment maximizing surface length for interference fixation. This positionally optimizes the interference screw fixation of the graft. The tendon is placed in a specific orientation relative to each bone block with the positional orientation of the bone blocks on the tendon being reversed from each other. After creating the tendon loop, the tendon loop is placed around each of the two plugs, tensioned on the graft table, sutured to the bone plugs through the suture bores and tied over the top of the tendons from one bore to another and from one tendon side to the other on each plug only. Each plug is independently sutured to the tendon separately from the other plug. When completely sutured, the graft remains on the graft table for the remainder of its pre-tensioning cycle—a minimum of 10 minutes at 10 lbs to 15 lbs of force.

When using multiple strands of tendons 50 to form a single replacement member as for example, a semitendinosus tendon, peroneus, tibialis anterior, tibialis posterior and/or gracilis tendon are extended between both of the bone blocks 30, 130. The tendon(s) 50 are preferably sutured to themselves at the distal and proximal ends only to create a tendon loop. One or more of the following tendons can be used as the replacement member: patellar, semitendinosus, gracilis, quadriceps, adductor magnus, the hamstrings, peroneus longus and hallucis longus. The tendons typically run from 180 mm to 300 mm in length and when recovered are fresh frozen or freeze dried after cleaning for preservation for use in the B-T-B assembly. The tendon can be sterilized with radiation dosages, gas or chemical means as is well known in the art. As such the tendon structure or member combining one or more of the above noted tendons will connect the two bone blocks.

Still further embodiments of the invention may substitute or combine man made or artificial fibers or human tissue instead of tendons or bone blocks for use as the ligament replacement.

The completed graft is inserted into the tibial tunnel and drawn through it and to the proximal end of the femoral tunnel with the suture 62 in the femoral bone plug. Once placed, a femoral interference screw is inserted with a screwdriver securing the femoral end of the graft.

The proper tension is then applied to the graft by tensioning the suture 62 on the tibial bone block or side. A driver and a headless interference screw are then inserted through the tibial tunnel for driving the screw along the curved exterior surface 36 of the bone block 30 crushing the cancellous bone with the cortical end portion 34 forming a stop so that the screw can not back out. In affixing the composite graft 10 within a bone tunnel, contact between an interference screw and the tendon 50 should be avoided so as not to cut or tear the tendon which is why the tendon is located in the channels 38. Once both plugs are secured, the insertion and tensioning sutures are removed from their respective bone plugs and the incisions are closed.

It is believed that 200 Newtons should be the minimum standard for pull out force, even though staples are currently being used by some medical graft providers with a 50 Newtons pull out force.

The unique features of allograft bone that make it desirable as a surgical material are, its ability to slowly resorb and be remolded or integrated into the space it occupies while allowing the bodies own healing mechanism to restore the repairing bone to its natural shape and function. The second feature is the high mechanical pull out strength arising from the interference screw used with a bone block having a cortical end cap. Thus a means of accelerating the rate of biointegration of cancellous bone would improve the rate of healing and benefit the recipient patient. The bone blocks may also be surface demineralized to increase osteoinductiveness. Such demineralization is generally undertaken to a depth of 30 to 80 microns and most preferably to a depth of 50 to 60 microns.

It is well known that bone contains osteoinductive elements known as bone morphogenetic proteins (BMP). These BMP's are present within the compound structure of cortical bone and are present at a very low concentrations, e.g. 0.003%. The BMP's are present in higher concentrations in the cancellous bone portion. BMP's direct the differentiation of pluripotential mesenchymal cells into osteoprogenitor cells which form osteoblasts. The ability of freeze dried demineralized bone to facilitate this bone induction principle using BMP present in the bone is well known in the art. However, the amount of BMP varies in the bone depending on the age of the bone donor and the bone processing. Based upon the work of Marshall Urist as shown in U.S. Pat. No. 4,294,753, issued Oct. 13, 1981 and as described and shown in Clinical Orthopaedics and Related Research 55, November-December 1967 in an article entitled “The Accessibility of the Bone Induction Principle in Surface-Decalcified Bone Implants” by Dubuc and Urist, the proper demineralization or surface demineralization of cortical bone will expose the BMP and present these osteoinductive factors to the surface of the demineralized material rendering it significantly more osteoinductive. The removal of the bone mineral leaves exposed portions of collagen fibers allowing the addition of BMP's and other desirable additives to be introduced to the demineralized outer treated surface of the bone structure and thereby enhances the healing rate of the cortical bone in surgical procedures. In cancellous bone the structure is not as dense as cortical bone exposing the naturally occurring BMP's rendering the entire structure with biological properties similar to fully demineralized bone (DBM).

It is also possible to add one or more rhBMP's to the bone block by soaking and being able to use a significantly lower concentration of the rare and expensive recombinant human BMP to achieve the same acceleration of biointegration. The addition of other useful treatment agents such as vitamins, hormones, antibiotics, antiviral and other therapeutic agents could also be added to the bone block.

Any number of medically useful substances can be incorporated in the bone block and tendon assembly by adding the substances to the assembly. Such substances include collagen and insoluble collagen derivatives, hydroxyapatite and soluble solids and/or liquids dissolved therein. Also included are antiviricides such as those effective against HIV and hepatitis; antimicrobial and/or antibiotics such as lysostaphin, triclosan, erythromycin, bacitracin, neomycin, penicillin, polymyxin B, tetracycline, viomycin, chloromycetin and streptomycin, cefazolin, ampicillin, azactam, tobramycin, clindamycin, gentamycin and silver salts.

It is also envisioned that amino acids, peptides, vitamins, co-factors for protein synthesis; hormones; endocrine tissue or tissue fragments; synthesizers; enzymes such as collagenase, peptidases, oxidases; polymer cell scaffolds with parenchymal cells; angiogenic drugs and polymeric carriers containing such drugs; collagen lattices; biocompatible surface active agents, antigenic agents; cytoskeletal agents; cartilage fragments, living cells, cell elements such as chondrocytes, red blood cells, white blood cells, platelets, blood plasma, bone marrow cells, mesenchymal stem cells, pluripotential cells, osteoblasts, osteoclasts, fibroblasts, epithelial cells and entothial cells, natural extracts, tissue transplants, bioadhesives.

In particular, the use of growth factors such as transforming growth factor (TGF-beta), insulin growth factor (IGF-1), platelet derived growth factor (PDGF), fibroblast growth factor (FGF)(Numbers 1-23) and variants thereof, platelet derived growth factor (PDGF), vascular endothelial growth factor (VEGF), osteopontin; growth hormones such as somatotropin; cellular attractants and attachment agents; fibronectin; immuno-suppressants; permeation enhancers, e.g. fatty acid esters such as laureate, myristate and stearate monoesters of polyethylene glycol, enamine derivatives, alpha-keto aldehydes can be added to the composition.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A sterile bone-tendon-bone assembly comprising:
a first allograft bone block constructed with a cancellous portion and a cortical end portion, said bone block defining an outer curved surface, with two opposing longitudinal arcuate grooves cut into the exterior surface which will allow a tendon replacement member to be wrapped around the bone block, a second allograft bone block having the same construction as the first bone block with said tendon replacement member extending around said second bone block in said exterior longitudinal arcuate grooves, said second bone block being in a reversed orientation to said first bone block.

2. A sterile bone-tendon-bone assembly as claimed in claim 1 wherein said first and second bone blocks central have a plurality of suture holes drilled radially through each of said bone blocks through said longitudinal grooves.

3. A sterile bone-tendon-bone assembly as claimed in claim 1 wherein the distance between the bottom portion of said two opposing arcuate grooves runs between about 3.0 mm to about 6.0 mm.

4. A sterile bone-tendon-bone assembly as claimed in claim 1 wherein said first and second bone block constructed of allograft bone are taken from a tissue source with a cortical end cap and cancellous body.

5. A sterile bone-tendon-bone assembly as claimed in claim 1 wherein said first and second bone block are not allograft bone but are constructed of artificial materials that are suitably implantable in humans.

6. A sterile bone-tendon-bone assembly as claimed in claim 1 wherein said tendon replacement member comprises at least one tendon taken from a group of tendons consisting of a semitendinous tendon, a patellar tendon, gracilis tendon, quadriceps tendon, adductor magnus tendon, peroneus tendons, tibialis tendons, hallucis Achilles tendon, or tendon like material (fascia lata).

7. A sterile bone-tendon-bone assembly as claimed in claim 1 wherein at least one bone block is surface demineralized to a depth of 30 to 80 microns.

8. A sterile bone-tendon-bone assembly as claimed in claim 4 wherein a tissue source for at least one of said bone blocks is taken from an area of the ilium.

9. A sterile bone-tendon-bone assembly as claimed in claim 1 wherein said tendon replacement member is taken from a group of tendons consisting of a semitendinosus tendon, peroneus longus, tibialis anterior, tibialis posterior, or any other suitably sized tendon or ligament material with required strength and size.

10. A sterile bone-tendon-bone assembly as claimed in claim 1 wherein the distance between the bottom portion of said two opposing arcuate grooves runs between about 3.8 mm to about 4.2 mm.

11. A sterile bone-tendon-bone assembly as claimed in claim 1 wherein said tendon replacement member comprises a gracilis tendon.

12. A sterile bone-tendon-bone assembly as claimed in claim 1 wherein said tendon replacement member is a biocompatible synthetic material.

13. A sterile bone-tendon-bone assembly as claimed claim 1 wherein said bone block includes an additive taken from a group of additives consisting of living cells, cell elements such as chondrocytes, red blood cells, white blood cells, platelets, blood plasma, bone marrow cells, mesenchymal stem cells, pluripotential cells, osteoblasts, osteoclasts, fibroblasts, epithelial cells and entothial cells, natural extracts, and tissue transplants.

14. A sterile bone-tendon-bone assembly as claimed in claim 1 wherein each bone block includes an additive taken from a group of additives consisting of transforming growth factor (TGF-beta), insulin growth factor (IGF-1), platelet derived growth factor (PDGF), fibroblast growth factor (FGF)(Numbers 1-23) and variants thereof, platelet derived growth factor (PDGF), vascular endothelial growth factor (VEGF), osteopontin, somatotropin, and growth hormones.

15. A sterile bone-tendon-bone assembly comprising:
first and second arcuate allograft bone blocks with a cancellous body and an integral cortical end portion, each bone block defining opposing longitudinal channels cut in its outer surface, a tendon replacement member extending between said first and second bone blocks and seated in said channels of said first and second bone blocks, a plurality of suture holes cut through said channels to receive sutures holding said tendon replacement member in a secured relationship to said bone blocks; said second bone block being in a reversed orientation of said first bone block, and at least one passing suture bore extending through at least one said bone block transverse said plurality of channel suture holes.

16. A sterile bone-tendon-bone assembly as claimed in claim 15 wherein said tendon replacement member comprises a loop structure.

17. A sterile bone-tendon-bone assembly as claimed in claim 15 wherein said tendon replacement member comprises a plurality of strands.

18. A sterile bone-tendon-bone assembly as claimed in claim 15 wherein at least one of said bone blocks is constructed of allograft bone which is surface demineralized in a range of 30 to 80 microns.

19. A sterile bone-tendon-bone assembly as claimed in claim 15 wherein said first and second bone block constructed of allograft bone are taken from an iliac crest, ilium, or other suitable anatomic tissue location.

20. A sterile reconstructed cruciate tendon assembly comprising:
first and second allograft bone blocks taken from an iliac crest or ilium, each block having a cancellous body and a cortical end portion; a plurality of opposing longitudinal channels cut in said curved outer surface of each of said bone blocks running the length of the bone block, a plurality of through going suture bores in each bone block positioned transverse to the longitudinal axis of each bone block and opening in said longitudinal channels, a linear replacement member extending between said first and second bone blocks mounted in said bone block longitudinal channels and attached alongside each of said first and second bone blocks with sutures which extend through said through going suture bores in said bone block, said second allograft bone block being positioned on said tendon replacement member in a reversed position from said first allograft bone block.

21. The sterile bone-tendon-bone assembly of claim 20 wherein each bone block defines at least one through going passing suture bore opening on the curved outer surface of the bone block positioned between said opposing longitudinal channels.
